(19) United States
(12) Patent Application Publication
(10) Pub. No.: US 2021/0038682 A1
(43) Pub. Date: Feb. 11, 2021

(54) MODULATORS OF SMALL CONDUCTANCE CALCIUM ACTIVATED K+ CHANNELS AND PHARMACEUTICAL COMPOSITIONS FOR USE IN THE TREATMENT OF LESIONAL VESTIBULAR DISORDERS

(71) Applicants: CENTRE NATIONAL DE LA RECHERCHE SCIENTIFIQUE (CNRS), Paris (FR); UNIVERSITE D'AIX-MARSEILLE (AMU), Marseille (FR)

(72) Inventors: Brahim Tighilet, Marseille (FR); Christian Chabbert, Nîmes (FR); Christiane Mourre, Marseille (FR); David Pericat, Greasque (FR); Jacques Léonard, Marseille (FR)

(73) Assignees: CENTRE NATIONAL DE LA RECHERCHE SCIENTIFIQUE (CNRS), Paris (FR); UNIVERSITE D'AIX-MARSEILLE (AMU), Marseille (FR)

(21) Appl. No.: 16/980,334

(22) PCT Filed: Mar. 13, 2019
(86) PCT No.: PCT/EP2019/056342

§ 371 Date: Sep. 11, 2020

(30) Foreign Application Priority Data
Mar. 13, 2018 (EP) 18161367.0
Oct. 18, 2018 (EP) 18020517.1

(51) Int. Cl. A61K 38/12; A61P 1/08

(52) U.S. Cl. CPC A61K 38/12; A61P 1/08

(57) ABSTRACT

The invention relates to a modulator of small conductance calcium activated K+ channels of the vestibular nuclei cells for use in the treatment of a lesional vestibular disorder in a patient in need thereof, and to a pharmaceutical composition comprising such modulator, for such a use.

### TECHNICAL FIELD

The invention relates to modulators of small conductance calcium activated K⁺ channels and to pharmaceutical compositions comprising such modulators for use in the treatment of a lesional vestibular disorder in patients in a need thereof.

### BACKGROUND OF THE INVENTION

The balanced sensory inputs arising from the vestibular end organs located in the two inner ears are essential to achieve high fidelity signaling of any head accelerations. Central integration of these vestibular inputs with those of vision and proprioception allows the vestibular system to permanently react to accelerations of the head in setting appropriate motor responses to maintain our posture and balance. Sudden alteration of the sensory inputs arising from peripheral vestibular receptors evokes characteristic vestibular syndrome characterized by a cascade of functional disorders that includes postural imbalance at rest and, during movement, spontaneous nystagmus and oscillopsia, associated to cognitive and neurovegetative disorders. These vestibular disorders result from alteration of the vestibulo spinal and vestibulo oculomotor reflexes, and modulations along the vestibulo cerebellar and cortical pathways. The vestibular syndrome may be especially pronounced in human under unilateral vestibular impairments such as labyrinthine fistula, vestibular neuritis or Meniere disease. In human, as in animal models of vestibular disorders of peripheral origin, i.e. vestibular peripheral vestibulopathies, the vestibular syndrome is generally composed of several phases, the amplitude of which depends on the type, stage and severity of the peripheral damage. The “acute” phase characterizes the period in which static disorders (posturo-locomotor symptoms and spontaneous nystagmus at rest) are the most prominent. This phase generally lasts several hours, but may extend to days. Subsequently, spontaneous decline of the vestibular symptoms amplitude takes place through a phenomenon referred to “vestibular compensation”. In its early phase, that takes place within days following the vestibular insult, a cascade of complex biological changes occurs in the brain stem vestibular nuclei in order to counteract the alteration of the functional homeostasis. Over a longer period, that may last several months, remaining vestibular disorders progressively disappear, leaving place to a “compensated” state. Both the early and late compensation processes concur to restore the posture and balance, though in most cases dynamic deficits never fully disappear.

Based on the above, there remains a need for discovering active ingredients and pharmaceutical compositions that allow to reach compensated states limiting the vestibular disorders related, in particular, to the “acute” phase. In other words, there remains a need for discovering active ingredients and pharmaceutical compositions that allow treating the vestibular disorders and, in particular, the “acute” phase of said disorders, in an efficient way, notably treating static disorders and complex biological changes in the brain stem vestibular nuclei.

### SUMMARY OF THE INVENTION

In accordance with a first aspect, the invention relates to a modulator of small conductance calcium activated K⁺ channels of vestibular nuclei cells for use in the treatment of a lesional vestibular disorder in a patient in need thereof.

Preferentially, —the modulator is for use in the treatment of a lesional peripheral vestibular disorder; —the disorder is selected from the group consisting of vertigo, nystagmus, balance unsteadiness and loss of muscular tonus; —the modulator is an inhibitor of small conductance calcium activated K⁺ channels; —the inhibitor is apamin, ULC1684, tapamin, tapamin-2, NS8593, leiurotoxin I, POᵢ, PO₂ and PO₅, tityus κ, BmSKTx1, tubocurarine, atracurium, dequalinium, or AG525E1; —the inhibitor is apamin; —the modulator is administered in an amount ranging from 0.1 mg/kg and 0.5 mg/kg; is apamin that is administered at a level of approximately 0.3 mg/kg; —the modulator is an activator of small conductance calcium activated channels; the activator is 1-EBIO, SKA-31, Chlorzoxazone, CyPPA, SKA-111, NS309 or NS13001; —the small conductance calcium activated channels comprise three subunits SK1, SK2 and SK3 and the modulator inhibits or activates said channels in acting on at least one of these subunits; —the modulator is acting on the SK2 subunit of the small conductance calcium activated channels; —the modulator is administered between 30 min and 5 h and, preferentially, between 30 min and 2 h and, more preferentially, between 30 min and 1 h after the insult.

In accordance with a second aspect, the invention relates to a pharmaceutical composition comprising at least a modulator as above, and at least one pharmaceutically acceptable excipient, for use in the treatment of a lesional vestibular disorder in a patient in need thereof.

Preferentially, the composition is administered by intraperitoneal route.

### BRIEF DESCRIPTION OF THE FIGURES

Other features and aspects of the present invention will be apparent from the following description and the accompanying drawings, in which:

FIG. 1 illustrates an experimental protocol elaborated for studying the effects of unilateral vestibular neurectomy (UVN) on the SK channels expression in vestibular nuclei (VN) and related structures (n=6 animals per group) and the consequences of apamin intraperitoneal injection on the time course of oculomotor and posturo-locomotor function recovery (n=4 animals per group);

FIG. 2 illustrates the [¹²⁵I] apamin binding sites in the cat brainstem, the illustrations being provided for serial sections collected from the rostral (6) to the caudal (10) parts of the brainstem;

FIG. 3 illustrates the effects of a unilateral vestibular neurectomy on the density of [¹²⁵I] apamin binding sites in the vestibular nuclei;

FIG. 4 illustrates the effects of a unilateral vestibular neurectomy on the density of [¹²⁵I] apamin binding sites in the three parts of the inferior olive: IOD, IOM, and IOP, dorsal accessory, medial accessory, and principal nucleus of the inferior olive, respectively; and

FIG. 5 illustrates the behavioral recovery time-course, that can be accelerated according to the apamin treatment after unilateral vestibular neurectomy. The curves in A illustrate the mean postoperative recovery of the support surface in the two experimental groups of cats (UVN-NaCl and UVN-apamin). The curves in B illustrate the time-course (abscissae) of disappearance of horizontal spontaneous nystagmus (HSN) frequency (ordinates) for each group of vestibular deafferented cats at different postoperative days. The curves in C illustrate the maximal performance (Max P.) that is defined as the highest beam rotation speed that did not lead to a fall on four consecutive crossings;

FIG. 6 illustrates the effects of an apamin treatment on vestibular syndrome severity (FIG. 6A), animal velocity (FIG. 6B), total distance covered (FIG. 6C), immobility time (FIG. 6D) and animal path shape (normalized meander) (FIG. 6E) as a function of time in days. In FIG. 6 “*” is indicative of a significant difference (p<0.05), “**” is indicative of a very significant difference (p<0.01), and “***” in indicative of highly significant difference (p<0.001) compared to a negative control group, two-way ANOVA; and

FIG. 7 illustrates the effects of AG525E1 (10 mg/kg) and NS8593 (30 mg/kg) treatments on vestibular syndrome severity (FIG. 7A, FIG. 7C) and immobility time (FIG. 7B) as a function of time in days. In FIG. 7 “*” is indicative of a significant difference (p<0.05) compared to a negative control group, two-way ANOVA.

### DETAILLED DESCRIPTION OF THE INVENTION

As used herein, the terms “treating”, “treatment”, and “therapy” refer to a curative or symptomatic therapy. Accordingly, the aim of the invention is to provide a relieve of the vestibular disorders or an amelioration of the patient's condition by alleviating the symptoms (nystagmus, postural imbalance, erroneous sensation of movement, dizziness) and promoting the vestibular functional recovery (gaze stabilization, static and dynamic balance).

The terms “vestibular disorder” or “vestibular syndrome” refer to a disorder of the vestibular system which includes the parts of the inner ear and brain that process the sensory information involved with controlling balance and eye movements. If an injury or a disease damage these areas, vestibular disorders appear.

The term “lesional vestibular disorder” or “lesional vestibular deficit” or “lesional vestibular syndrome” refers to vestibular disorders wherein lesions on inner ear cells and/or vestibular nerve are present or will appear during the disorder time course. In this case, the functionality of the vestibule is impaired. Lesional vestibular disorders include: —vestibular disorders wherein an infection inflames the inner ear and or the vestibular nerve inducing reversible and/or irreversible damages, one example of conditions from this group is vestibular neuritis; —vestibular disorders wherein inner ear fluid levels are affected (abnormalities in the quantity, composition, and/or pressure of the endolymph), these disorders usually develop lesions during the disease time course, such as Meniere's disease and secondary endolymphatic hydrops; —vestibular disorders induced by insults or lesions of the vestibular end-organs, such as vertigo causes by local ischemia, excitotoxicity. Examples of lesional vestibular disorders that are contemplated by the invention include but are not limited to neuritis, viral neuronitis, labyrinthitis, viral endolymphatic labyrinthitis, drug-induced ototoxicity, Meniere's disease, endolymphatic hydrops, head trauma with lesional vestibular deficits, labyrinthine haemorrhage, chronic or acute labyrinthine infection, serious labyrinthine, barotraumatism, autoimmune inner ear disease, presbyvestibulia, toxic vestibular impairments.

The different phases of the vestibular syndrome are supported by major changes in the excitability of the vestibular secondary neurons (VSNs) within the brain stem vestibular nuclei (VN). Unilateral vestibular lesion abruptly depresses the spontaneous resting activity of the VSNs on the deafferented side, while it conversely increases the excitability of those located in the VN, contralaterally to the lesion. These opposite effects rely on the removal of the excitatory glutamatergic inputs from the vestibular primary neurons (VPNs) on the VSNs of the deafferented side. They also result from the runaway of the excitability of the VSNs on the side opposed to the insult, due to the decreased weight of the commissural inhibition exerted by the ipsilateral VSNs. The depression of the ipsilateral VSNs spontaneous discharge is furthermore accentuated by the increased weight of the commissural inhibition exerted by the contralateral VSNs. This situation results in an imbalance of activity between opposite VNs. Subsequently, the discharge activity of the VSNs on the deafferented side spontaneously recovers. This phenomenon is first observed at distance (several weeks) from the triggering insult in cat models of unilateral labyrinthectomy (UL) or unilateral vestibular neurectomy (UVN), and later demonstrated to be already present over the first days following the peripheral damage in slices preparations of VN in rat models of UL.

This invention provides modulators of small conductance calcium activated K⁺ channels and pharmaceutical compositions comprising such modulators for use in the treatment of a lesional vestibular disorders in patients in need thereof.

As used herein, the term “modulator” refers to any molecule, agent or compound that increases or decreases small conductance calcium activated K⁺ channels activity, said modulator being an activator or an inhibitor as defined herein below.

The patients are mammals and, more particularly, humans.

The sudden and unilateral loss of peripheral vestibular inputs alters the expression of SK-type channels in the brain stem vestibular nuclei. This process participate into the acute vestibular syndrome as well as the compensatory mechanisms. The administration of at least one modulator of small conductance calcium activated K⁺ channels displays a significant antivertigo effect.

The modulator of small conductance calcium activated K⁺ channels is an inhibitor or an activator of the activity of such small conductance activated K′ channels.

If the modulator is an inhibitor of such channels, it has direct antagonist or negative modulation effects on the SK channels.

The term “inhibitor” as used herein, refers to an agent that has antagonist or negative modulation effects on the SK channels. In particular, an inhibitor according to the invention can be a molecule selected from a peptide, a peptide mimetic, a small organic molecule, an antibody, an aptamer, a polynucleotide and a compound comprising such a molecule or a combination thereof. Preferably, said inhibitor is a peptide or a small organic molecule.

For example, the inhibitor is apamin (an 18 amino acid peptide neurotoxin found in apitoxin—CAS number 24345-16-2); ULC1684 (also named 6,12,19,20,25,26-Hexahydro-5,27:13,18:21, 24-trietheno-11,7-metheno-7H-dibenzo [b,n] [1,5,12,16]tetraazacyclotricosine-5,13-diium dibromide, see Campos Rosa, J., Galanakis, D., Ganellin, C. R., Dunn, P. M., Jenkinson, D. H., 1998. “Bis-quinolinium cyclophanes:6,10-diaza-3 (1,3), 8 (1,4)-di-benza-1,5 (1,4)diquinolinacyclodecaphane (UCL 1684), the first nanomolar, non-peptidic blocker of the apamin sensitive Ca²⁺ activated K⁺ channel”. J. Med. Chem. 41, 2-5.); tamapin (see Pedarzani P, D'hoedt D, Doorty K B, Wadsworth J D, Joseph J S, Jeyaseelan K, Kini R M, Gadre S V, Sapatnekar S M, Stocker M, Strong P N (2002). “Tamapin, a venom peptide from the Indian red scorpion (Mesobuthus tamulus) that targets small conductance Ca2+-activated K+ channels and after hyperpolarization currents in central neurons”. J. Biol. Chem. 277 (48): 46101-9.); tamapin-2 (an isoform of tamapin, in which the tyrosine is replaced by a histidine); NS8593 (also named N-[(1R)-1, 2, 3, 4-Tetrahydro-1-naphthalenyl]-1H-benzimidazol-2-amine hydrochloride); leiurotoxin I (also named Scyllatoxin); POᵢ, PO₂, PO₅, PO₅-NH2; tityus κ (see Legros et al. “Characterization of a new peptide from Tityus serrulatus scorpion venom which is a ligand of the apamin-binding site”, FEBS Letters 390 (1996) 81-84); BmSKTx1 (see Xu et al. “A novel scorpion toxin blocking small conductance Ca2+ activated K+ channel.”, Toxicon. 2004 Jun. 15; 43(8):961-71.); tubocurarine (also known as d-tubocurarine or 6,6′-dimethoxy-2,2′,2′-trimethyltubocuraran-2,2′-diium-7′,12′-diol, cas number 57-95-4); atracurium (also named 3-[1-[(3,4-diméthoxyphényl)méthyl]-6,7-diméthoxy-2-méthyl-3,4-dihydro-1H-isoquinoléine-2-yl]propanoate de 5-[3-[1-[(3,4-diméthoxyphényl)méthyl]-6,7-diméthoxy-2-méthyl-3,4-dihydro-1H-isoquinoléine-2-yl]propanoyloxy]pentyle); dequalinium (also named 1,1′-decane-1,10-diylbis(4-amino-2-methylquinolinium) decyl]-2-methyl-4-quinolin-1-iumamine dichloride) or compound AG525E1 (1,1′-(propane-1,3-diyl)-bis-(6, 7-dimethoxy-2-methyl-1,2,3,4-tetrahydroisoquinoline)).

Preferentially, the inhibitor is apamin.

If the modulator is an activator of such channels, it has direct agonist or positive modulation effects on the SK channels.

The term “activator” as used herein, refers to an agent that has agonist or positive modulation effects on the SK channels. In particular, an inhibitor according to the invention can be a molecule selected from a peptide, a peptide mimetic, a small organic molecule, an antibody, an aptamer, a polynucleotide and a compound comprising such a molecule or a combination thereof. Preferably, said activator is a small organic molecule.

For example, the activator is 1-EBIO (1-Ethyl-2-benzimidazolinone), SKA-31 (Naphtho[1,2-d]thiazol-2-ylamine), Chlorzoxazone, CyPPA (N-Cyclohexyl-N-[2-(3,5-dimethyl-pyrazol-1-yl)-6-methyl-4-pyrimidinamine), SKA-111 (5-Methylnaphtho[1,2-d]thiazol-2-amine), NS309 (6,7-Dichloro-1H-indole-2,3-dione 3-oxime) or NS13001 ((4-Chlorophenyl) [2-(3,5-dimethylpyrazol-1-yl)-9-methyl-9H-purin-6-yl]amine).

The term “antibody” is used in the broadest sense, and covers monoclonal antibodies (including full-length monoclonal antibodies), polyclonal antibodies, multispecific antibodies, chimeric antibodies, antibodies fragment and humanized antibodies, so long as they exhibit the desired biological activity. Antibody fragments comprise a portion of a full length antibody, generally an antigen binding or variable region thereof. Examples of antibody fragments include Fab, Fab′, F(ab′)2, and Fv fragments, diabodies, linear antibodies, single-chain antibody molecules, single domain antibodies (e.g., from camelids), shark NAR single domain antibodies, and multispecific antibodies formed from antibody fragments.

For example, the immediate modulatory effect of apamin could combine a stimulatory action on the excitatory type I VSNs on the injured side, whose excitability is greatly reduced after UVN, with a simultaneous action on type I inhibitory VSNs of the opposite side. The antivertigo action of apamine could result from a rebalancing of the spontaneous activity between opposite VNs. This hypothesis is interesting because, conversely to a vestibulo-depressant, aiming at reducing the imbalance between the opposing VNs by simultaneous inhibitory actions, the excitatory action of apamine would reach similar result, while exacerbating hyperexcitability. Acceleration of the vestibular compensation under apamin administration, observed in the acute phase of the compensation and extending to its late phase is an illustration of such a phenomenon. Beyond apamin by itself, other molecules with direct antagonist or negative modulation effects on SK channels, such as the precited UCL1684, tamapin, NS8593, AG525E1 inhibitors are also efficient in alleviating the vestibular syndrome. Given the diversity and complexity of the neuronal populations present in the vestibular nuclei, including excitatory versus inhibitory neurons acting both locally and on the other side, the agonization of SK channels by direct agonists or positive modulators, such as the precited 1-EBIO, SKA-31, Chlorzoxazone, CyPPA, SKA-111, NS309 and NS13001 activators, can also promote benefit through a rebalancing spontaneous activity between opposite vestibular nuclei.

The pharmaceutical composition of the present invention comprises at least one modulator of small conductance calcium activated K⁺ channels of the vestibular nuclei cells such as neurons and other cell types, and at least one pharmaceutically acceptable excipient for use in the treatment of a vestibular disorder in patient in need thereof. The modulator is an inhibitor or an activator of the small conductance calcium activated K⁺ channels.

The calcium activated K⁺ channels are potassium channels gated by calcium, or that are structurally or phylogenetically related to calcium gated channels. These channels are divided into three subtypes: (i) the large conductance or BK channels, (ii) the intermediate conductance or IK channels, and (iii) the small conductance or SK channels. This family of ion channels is in particular activated by intracellular Ca²⁺. In particular, SK channels are members of the voltage insensitive calcium-activated potassium channels family. Upon elevation of the cytosolic calcium concentration, the channels open, allowing K⁺ ions to leave the cell as a function of the K⁺ equilibrium potential. Their activation leads to the cell hyperpolarization. More specifically, the SK channels regulate neuronal excitability by contributing to the slow component of synaptic after hyperpolarization (AHP). Their activation or up regulation is expected to limit the firing frequency of repetitive action potentials. Regarding these specific gating properties, it can be elaborated on the expected functional consequences of the SK channels up regulation in the VNs depending on the cell type and the side considered. In the brain stem VNs, it can be assumed that the observed up regulation of the SK channels expression may especially take place in microglia cells, astrocytes and VSNs, three cell types previously reported to express this SK channel subtypes.

In a specific embodiment, the small conductance calcium activated K⁺ channels comprises three subunits SK1, SK2 and SK3 and the modulator inhibits or activates at least one of these subunits. In a more preferred embodiment, the modulator inhibits or activates the subunits SK2 or SK3.

The pharmaceutical composition is adapted for use in the treatment of peripheral vestibular disorders, i.e. vestibular peripheral vestibulopathies. The peripheral vestibular disorders are caused by dysfunction in the semicircular channels, the vestibule (utricle and saccule), or the vestibular nerve. As an example, the main causes of peripheral vestibular syndrome are Benign Positional Paroxysmal Vertigo (BPPV), Meniere's disease, vestibular neuritis and labyrinthitis. For example, vestibular disorders are selected from the group consisting of vertigo/dizziness, nystagmus, balance unsteadiness, loss of muscular tonus, often accompanied by neurovegetative manifestations such as nausea, vomiting, and salivation, and perceptive-cognitive manifestations such as alteration of the body schema, subjective vertical perception, spatial disorientation.

In another preferred embodiment, the modulator of small conductance calcium activated K⁺ channels is apamin. Apamin is an 18 amino acid neurotoxin found in apitoxin, the venom of biting insects such as bees or wasps. This neurotoxin blocks the small-conductance calcium-activated potassium channels of the nervous system. It is used in medicine as an experimental treatment against Parkinson's disease.

According to a specific embodiment of the invention, the pharmaceutical composition can be administered by intraperitoneal route, intravenous and per os administrations, preferentially by intraperitoneal route. Apamin is administered, in the present invention, intraperitoneally once a day, during the three days following the UVN, in an amount ranging from 0.1 mg/kg to 0.8 mg/kg, preferentially from 0.1 mg/kg to 0.6 mg/kg, for example, from 0.1 mg/kg to 0.5 mg/kg , preferentially administered at a level of approximately 0.3 mg/kg or 0.6 mg/kg.

The pharmaceutical composition according to the invention, is in particular administered between 30 min and 5 h and, preferentially, between 30 min and 2 h and, more preferentially, between 30 min and 1 h after the insult or insult induction. The diagnosis of the vestibular disorder was assessed between 30 min and 5 h and, preferentially, between 30 min and 2 h and, more preferentially, between 30 min and 1 h after the insult or insult induction.

### METHODS AND EXAMPLES

Methods

The following methods were used for the implementation of the invention, and in the examples:

Animals

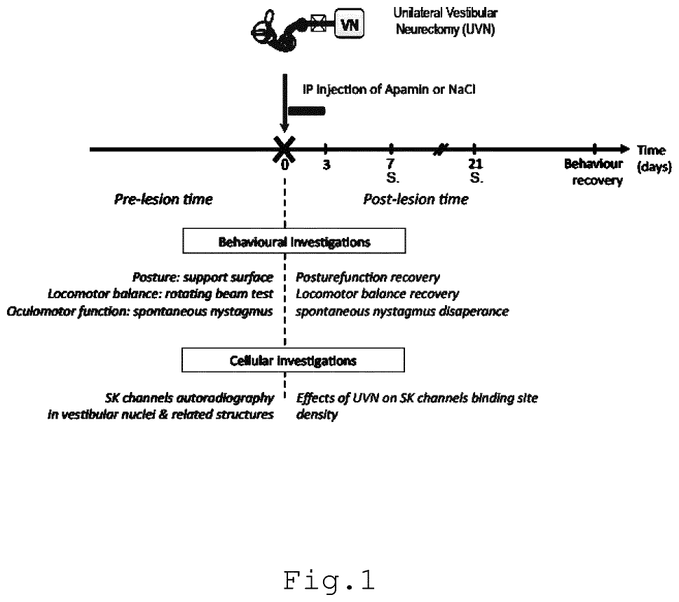

Experiments were performed on 18 adult domestic cats (3-5 kg) obtained from the “Centre d'élevage du Contigne” (Contigne, France). All experiments were carried out in line with the Animals (scientific procedures) Act, 1986 and associated guidelines, the European Communities Council Directive of 24 Nov. 1986 (86/609/EEC), and the National Institutes of Health guide for the care and use of laboratory animals (NIH publications No. 8023, revised 1978). Every attempt was made to minimize both the number and the suffering of animals used in this experiment. Cats were housed in a large confined space with normal diurnal light variations and free access to water and food. Eighteen animals were used for SK channels binding study in the vestibular nuclei and related brain stem structures. A group of intact animals (n=6) was used as control group. The remaining 12 cats were submitted to left UVN and killed at 2 survival times: 1 week (n=6) and 3 weeks (n=6). Survival times were selected from our previous behavioral and electrophysiological investigations in the cat, which had showed major postural deficits in acute cats (1 week) and nearly complete recovery in compensated animals (3 weeks). To determine the effects of apamin treatment on the time-course of the cats' recovery at a behavioral level, 8 additional UVN cats were used for this study, they received during the three post UVN days, an intraperitoneal injection of NaCl (n=4) or Apamin (n=4), (see FIG. 1).

Vestibular Neurectomy

A left side vestibular nerve section was performed under aseptic conditions through a dissecting microscope. Animals were first anaesthetized with Ketamine (20 mg/kg, i.m.; Rhone Poulenc, Merieux, France), received analgesic (Tolfedine, 0.5 ml, i.m.; Vetoquinol, Lure, France), maintained under fluothane anesthesia (2%) and were kept at physiological body temperature using a blanket. The vestibular nerve was sectioned on the left side at a post-ganglion level in order to leave the auditory division intact after mastoidectomy, partial destruction of the bony labyrinth, and surgical exposure of the internal auditory canal, (according to Xerri and Lacour, 1980). Animals were maintained under antibiotics for 7 days and analgesics for 3 days. Classical postural, locomotor, and oculomotor deficits displayed by the animals in the days following nerve transection were used as criteria indicating the effectiveness of the vestibular nerve lesion. Completeness of vestibular nerve section had already been assessed by histological procedures in previous studies (Lacour et al., 1976).

Tissue Preparation

Cats of each group were deeply anesthetized with ketamine dihydrochoride (20 mg/kg, IM, Merial, Lyon, France) and killed by decapitation; after removal from the skull, their rains were cut into several blocks containing the brainstem structures (VN and IO), and the blocks were rapidly frozen with CO₂ gas. Coronal sections (10-mm-thick) were cut in a cryostat (Leica, Reuil-Malmaison, France), thawed onto Superfrost ++ glass slides (Fisher Scientific, Elancourt, France), and stored at −80° C. until radio autography. Experiments were carried out blind; the group that the cats belonged to was unknown to the person conducting binding.

Apamin Binding Experiment

For binding experiments, tissue sections were incubated with highly radioactive apamin labeled with [¹²⁵I] (PerkinElmer) as previously described (Mourre et al., 1986). Brain sections were incubated with 25 pM [¹²⁵I] -apamin, at 4° C. in 100 mM Tris-Cl buffer, containing 0.5% bovine serum albumin (BSA), pH 7.4. Non-specific binding was assessed by adding a large excess of native apamin (Sigma, 0.1 μM) before adding [¹²⁵I]-apamin. Sections were incubated for 60 min and rinsed three times, each for 20 s, in the same buffer. The sections were rinsed a fourth time, for 20 s, in water. Dried sections were placed on Kodak BioMax MR films. Serial sections of one naive cat were added with experimental sections, serving as internal standards for labeling on the different films. Autoradiograms were exposed for 12 days to obtain unsaturated labeling and thus to allow the detection of increases or decreases in labeling. Films were then processed in a Kodak Industrex developer. Autoradiograms were analyzed and radioactivity quantified with NIH Image software. Plastic standards (Amersham) were used to calibrate [¹²⁵I] concentrations. Mean receptor density was calculated for each unilateral nucleus, using two to three measurements in each stereotaxic level for each animal. Non-specific binding was detected on autoradiograms of sections incubated with unlabeled 0.1 μM apamin, corresponding to around 15% of total binding. Specific binding was calculated as the difference between total and non-specific binding for a given area. Azur II stained sections were used for reference. Cat brainstem structures including each of the four main vestibular nuclei (medial, inferior, superior and lateral) and the three subdivisions of the inferior olive (the principal nucleus (IOP), medial accessory (IOM), and dorsal accessory (IOD) of the inferior olive) were identified and named using a cat brain atlas Berman's stereotaxic atlas (Berman, 1968).

Apamin Administration

Apamin (0.3 mg/kg, dissolved in NaCl 0.9%, Genepep, France) was injected intraperitoneally (i.p.) 30 min before each behavioral test. Systemic administration of apamin was chosen because it was found to cross the blood-brain barrier. The animals were allocated to two different subgroups (vehicle lesioned and apamin-lesioned (0.3 mg/kg). For each subgroup it is determined the effects of these drug treatments on the recovery of posturo-locomotor and oculomotor functions through adapted behavioral tests. The behavioral evaluation of the effects of apamin administration was done in blind condition.

Behavioral Investigations

Spontaneous nystagmus recovery: The spontaneous horizontal vestibular nystagmus induced by the UVN was recorded by videotracking of the eyes movement as previously detailed (Tighilet et al. 2006). The frequency of the horizontal spontaneous nystagmus was measured in the light as the number of quick phase beats towards the contralateral side relative to UVN in 10 sec (five repeated measures per animal per sampling time). Each recording session (duration=15 min) was located at the same period of the day (in the morning) in order to counteract possible variations due to the alertness state of the animals. Full recovery was achieved when the vestibular nystagmus totally disappeared in the light.

Posture recovery: The support surface measure serves to evaluate the postural stability of the animal. Posture deficits and recovery were evaluated by measuring the surface delimited by the four legs of the cat while standing erect at rest, without walking. Support surface is considered a good estimate of postural control since it reflects the cat's behavioral adaptation compensating the static vestibulospinal deficits induced by the vestibular lesion (Tighilet et al., 1995). As a rule, the surface was very small in the normal cat (about 50-100 cm²) and greatly increased in the days following unilateral vestibular lesion. To quantify the support surface, cats were placed in a device with a graduated transparent floor that allowed them to be photographed from underneath. Five repeated measurements were done for each cat tested at each postoperative time, and an average was calculated for each experimental session. The support surface was measured as the surface delimited by the four legs by an image analysis system (Canvas, 9™, Deneba software, Miami, Fla.). Data recorded after vestibular lesion were compared to pre-lesion values by using individual references, permitting each animal to act as its own control.

Equilibrium function recovery: Locomotor balance function was quantified using an adapted rotating beam experimental device (Xerri and Lacour, 1980). Two compartments (0.5×0.6×0.5 m) were connected by a horizontal beam (length: 2 m; diameter: 0.12 m). The beam, placed 1.2 m off the ground, can be rotated along its longitudinal axis with a constant angular velocity ranging from 0° to 588.4°/s (about 1.5 turn/s). Behavioral training on the rotating beam consisted in depriving the animals of food overnight before the first training session. Animals were conditioned to cross over the beam and were rewarded by a small piece of fish (or meat) placed in a small bowl in the target compartment. First crossings were made on the immobile beam and, thereafter, on the rotating beam. As a rule, rotation velocity of the beam was progressively increased after four consecutive trials without fall. Equilibrium function was thus quantified by measuring the highest speed of beam rotation that did not induce a fall. This maximal rotation speed determined the maximal locomotor balance performance (Max P.). Preoperative training on the rotating beam necessitated 6 to 10 periods depending on the cats. Training was stopped when the cats' Max P. was reached and stabilized at its highest level.

Statistical Analysis

Using Graphpad Prism6™ software, effects of lesion and of time course on apamin binding sites densities of the different groups were tested by means of two-way ANOVA, followed by adapted post-hoc tests between groups (Tukey's test) where P<0.05. All data were expressed as the mean±standard error of the mean (S.E.M.) and a P value of <0.05 was taken as the minimum level of significance. Because the number of cats by groups is four, the effects of lesion and of time course on behavioral performances were tested by means of nonparametric test, Kruskal-Wallis test, followed by adapted post-hoc tests between groups (Dunn's test) where P<0.05.

### EXAMPLES

Example 1: SK Channel Binding Sites Density Increases After Unilateral Vestibular Neurectomy

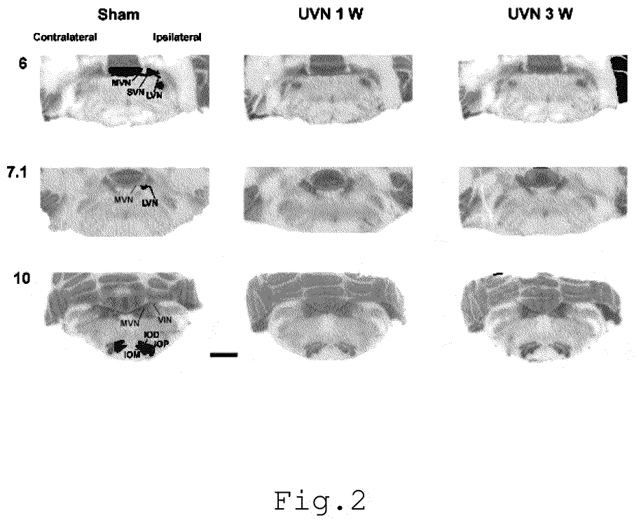

FIG. 2 illustrates the spatial distribution of apamin binding site density in representative serial frontal sections collected from the rostral (6) to the caudal (10) parts of the brainstem in a control cat (Sham) and in two representative cats killed 1 (1 W) or 3 (3 W) weeks after UVN. IVN, inferior vestibular nucleus; LVN, lateral vestibular nucleus; MVN, medial vestibular nucleus; SVN, superior vestibular nucleus; IOD, IOM, and IOP, dorsal accessory, medial accessory, and principal nucleus of the inferior olive, respectively. Bar: 1 mm. Coronal sections from representative sham and unilateral vestibular neurectomized cats showing increases in SK protein binding sensitive to apamin in the different structures of the brainstem on the contralateral and ipsilateral sides of lesion, 1 W or 3 W after unilateral vestibular neurectomy, as compared to the Sham (A). According to FIG. 2, in the control cat, the pattern of apamin binding was heterogeneous: intermediate to low levels of binding sites were found in the vestibular complex while higher levels were found in the inferior olive complex. Particularly, the medial and inferior vestibular nuclei contained apamin binding site densities higher than the superior and lateral vestibular nuclei.

Conclusion: These observations indicate that unilateral vestibular neurectomy induces a bilateral increase of the apamin binding sites density in the vestibular nuclei. This increase is more pronounced in the brain side contralateral to the insult.

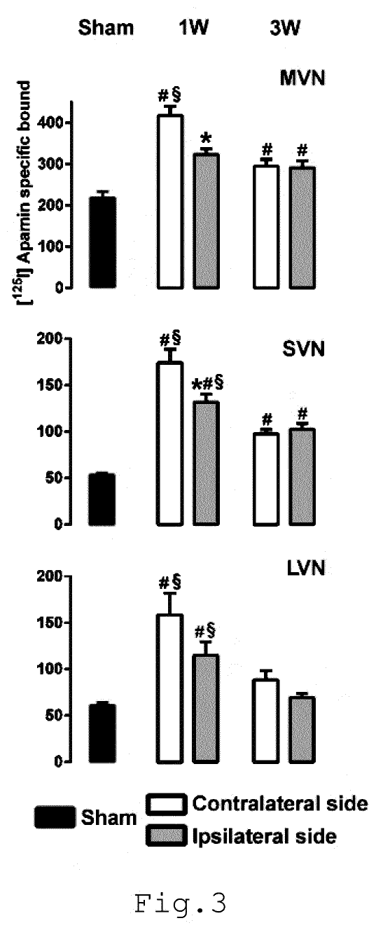

According to FIG. 3, the effects of a unilateral vestibular neurectomy on the density of [¹²⁵I] apamin binding sites in the vestibular nuclei is showed, in particular in medial (MVN), superior (SVN), and lateral (LVN) nuclei. Variations of binding level in sham cat group (n=4) and in cat groups 1 (1 W) and 3 (3 W) weeks after unilateral vestibular neurectomy (n=4 by group) is evaluated. Data are given as a value of binding on the ipsilateral and contralateral side of UVN in the structures. Results are expressed as mean values and S.E.M. of femtomole of [¹²⁵I] apamin specifically bound per milligram of protein from autoradiograms. “*”, P<0.05 (lesioned versus intact side in each group); “#”, P<0.05 (1 W or 3 W group versus sham group for each intact and lesioned side respectively), and “§”, P<0.05 (1 W group versus 3 W group for each intact and lesioned side respectively).

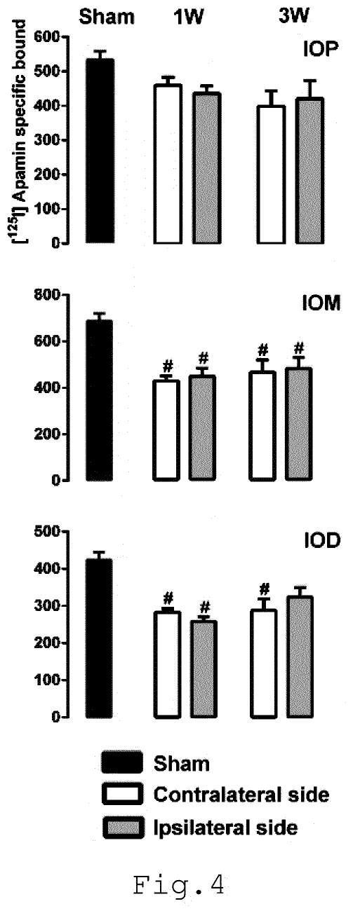

The Table 1 represents the levels of apamin binding sites in the vestibular complex and related nuclei. In particular, it is shown raw values of density measurements of labeling of binding sites of radioactive apamin protein. Such values supports results illustrated in FIGS. 3 and 4, and they are used for the static treatment explain below.

TABLE 1
Levels of Apamin binding sites in the vestibular complex and related nuclei

| | | Sham | | 1 W | | 3 W | |
|---|---|---|---|---|---|---|---|
| | | Controlateral | Unilateral | Controlateral | Unilateral | Controlateral | Unilateral |
| Medial vestibular | MVN | 218.80 ± 22.24 | 213.20 ± 20.55 | 417.90 ± 22.04 | 324.10 ± 12.61 | 295.40 ± 16.05 | 291.30 ± 16.86 |
| nucleus | | | | | | | |
| Superior vestibular | SVN | 48.82 ± 1.52 | 58.76 ± 2.56 | 180.30 ± 10.20 | 133.10 ± 5.92 | 97.51 ± 4.75 | 102.60 ± 6.13 |
| nucleus | | | | | | | |
| Lateral Vestibular | LVN | 55.46 ± 3.59 | 65.94 ± 4.82 | 115.40 ± 14.33 | 158.50 ± 23.62 | 69.65 ± 4.34 | 88.75 ± 9.83 |
| nucleus | | | | | | | |
| Inferior vestibular | VIN | 309.90 ± 21.69 | 325.10 ± 29.16 | 363.50 ± 19.28 | 351.20 ± 15.98 | 338.00 ± 24.36 | 373.00 ± 26.25 |
| nucleus | | | | | | | |
| Principal accessory | IOP | 554.00 ± 33.15 | 512.80 ± 37.93 | 461.40 ± 23.38 | 437.90 ± 21.51 | 400.20 ± 44.70 | 422.50 ± 51.51 |
| inferior olive | | | | | | | |
| Dorsel accessory | IOD | 411.20 ± 29.82 | 434.40 ± 30.06 | 282.10 ± 10.37 | 257.40 ± 12.24 | 287.30 ± 30.71 | 323.30 ± 25.47 |
| inferior olive | | | | | | | |
| Medial accessory | IOM | 667.90 ± 42.92 | 700.50 ± 50.11 | 423.90 ± 21.59 | 445.90 ± 33.58 | 462.90 ± 52.33 | 478.60 ± 46.98 |
| inferior olive | | | | | | | |
| Medial nucleus of | SM | 372.80 ± 36.08 | 407.90 ± 37.46 | 366.40 ± 32.09 | 365.70 ± 59.56 | 349.40 ± 54.91 | 291.80 ± 54.06 |
| the solitary tract | | | | | | | |
| Dorsal motor nucleus | DMGV | 458.60 ± 36.00 | 468.80 ± 46.38 | 527.30 ± 74.47 | 510.00 ± 83.87 | 441.30 ± 58.38 | 461.60 ± 36.53 |
| of the vagus | | | | | | | |

Data expressed in mean ± S.E.M. in fmol/mg of protein. Contralateral and ipsilateral: sides related to UVN, 1 W and 3 W: 1 and 3 weeks after UVN.

According to FIG. 3 and Table 1, in the medial vestibular nucleus (MVN), a two-way ANOVA revealed an interaction (F_{2,238}=4.20, F value is the result of the two ways ANOVA, 2 corresponds to n-1, n being the group number−sham/1 week/3 weeks—; 238 is the sample number, P<0.05) between the lesion and the postlesion time, an effect of postlesion time (F_{2,238}=31.59, P<0.01), and lesion (F_{1,238}=5.23, P<0.05) on the apamin binding site level.

One week after UVN, apamin binding site density was significantly increased on the ipsilateral side, compared to control (Tukey's, +52%, P<0.01). This increased apamin binding site density persisted 3 weeks after UVN. In the MVN contralateral to the lesion, the increase of apamin binding sites density in the 1 week postlesion group was significantly stronger than for the ipsilateral side compared to both the control and the 3 weeks post-lesion group (+90%, 37% respectively P<0.01). Moreover, at the one week post-lesion delay, the apamin binding sites density was significantly higher in the contralateral side than in the ipsilateral side (+29%, P<0.01). At the three weeks postlesion, the binding level increased bilaterally in both sides in comparison with the control group (Tukey's, +40%, P<0.05).

Concerning the superior vestibular nucleus (SVN, FIG. 3, Table 1), a two-way ANOVA showed an interaction (F_{2,174}=14.46, P<0.001) and an effect of post-lesion time (F_{2,174}=153.1, P<0.001), and a significant effect of lesion (F_{1,174}=4.95, P<0.003) on the apamin binding site level. One week after UVN, the apamin binding site density significantly increased in the ipsilateral and even more in the contralateral lesion side compared to control (+129 and +249% respectively, P<0.001). The binding level was significantly higher in the contralateral than the ipsilateral side (P<0.01). Moreover three weeks after UVN, apamin binding significantly increased in both sides compared to the control but significantly decreased compared to that observed at the one week post-lesion (P<0.01, P<0.05 respectively). No binding variation was observed at this time point between the two sides.

In the lateral vestibular nucleus (LVN, FIG. 3, Table 1), data analysis revealed an effect of lesion (F_{1,196}=5.64, P<0.05) and of post-lesion time (F_{2,196}=19.23, P<0.01) but no interaction between the two factors (F_{2,196}=0.86, NS), on the apamin binding. Tukey's test showed that one week after UVN, binding level significantly increased in contralateral (+140%, P<0.01) and ipsilateral (+108%, P<0.05) sides of the lesion compared to control like SVN and MVN. No significant binding variation was found between the two sides at this time point, even if the binding level in the contralateral side was higher than that in the ipsilateral side. Moreover, at 3 weeks post-UVN, the binding level was similar to the control. In contrast, in the inferior vestibular nucleus, no variation of apamin binding levels was found following UVN compared to control and whatever the post lesion time-points (Table 1).

Conclusion: Altogether, these observations confirm that the unilateral vestibular neurectomy induces a bilateral increase of the apamin binding sites density in the vestibular nuclei, that is more pronounced on the brain side contralateral to the insult. The increase in the density of the apamin binding sites is transient, as it is statistically significant 1 W, but not anymore 3 W after the UVN.

### Example 2: Nuclei Associated to the Vestibular Complex

According to FIG. 4, the effects of a unilateral vestibular neurectomy on the density of [¹²⁵I] apamin binding sites in the three parts of the inferior olive: IOD, IOM, and IOP, dorsal accessory, medial accessory, and principal nucleus of the inferior olive, respectively, are illustrated. The variations of binding level in sham cat group (n=4) and in cat groups 1 (1 W) and 3 (3 W) weeks after unilateral vestibular neurectomy (n=4 by group) are evaluated. Data are given as a value of binding on the ipsilateral and contralateral side of UVN in the structures.

Results are expressed as mean values and S.E.M. of femtomole of [¹²⁵I] apamin specifically bound per milligram of protein from autoradiograms. “#”, P<0.05 (1 W or 3 W group versus sham group for each intact and lesioned side respectively)

More specifically, according to FIG. 4 and Table 1, in the inferior olive (IO) complex, the UVN induced a decrease in apamin binding site level in the principal nucleus (IOP), medial accessory (IOM), and dorsal accessory (IOD) of the inferior olive. In all subdivisions, a two-way ANOVA revealed an effect of post lesion time (F_{2.92}≥5.32, P<0.01) but no significant interaction (F_{2.92}≤0.74, Not Significant) and no effect of lesion (F_{1.92}≤0.42, NS) on the apamin binding site level. Tukey's test showed that the apamin binding site density in the IOM and IOD was significantly reduced after 1 and 3 weeks after UVN (P<0.05) whatever the lesion side studied. In the principal nucleus of the inferior olive, only a tendency of a bilateral reduction of the binding level was observed after 1 and 3 weeks post-UVN. In the medial nucleus of the solitary tract and the dorsal motor nucleus of the vagus (Table 1), data analysis indicated that the UVN caused no difference of the levels of apamin binding sites whatever the post-lesion time and lesion sides (interaction and time, F_{2,44}≤0.58, NS; lesion F_{1,44}≤1.32, NS).

Conclusion:

Conversely to the vestibular nuclei, in the inferior olive complex, SK channel expression is significantly reduced at 1 and 3 weeks after the UVN, in the medial accessory and dorsal accessory (IOM and IOD), but not in the principal nucleus of the inferior olive. No difference in expression between the intact side and the injured side is observed at this level.

### Example 3: Functional Alterations Following Unilateral Vestibular Neurectomy

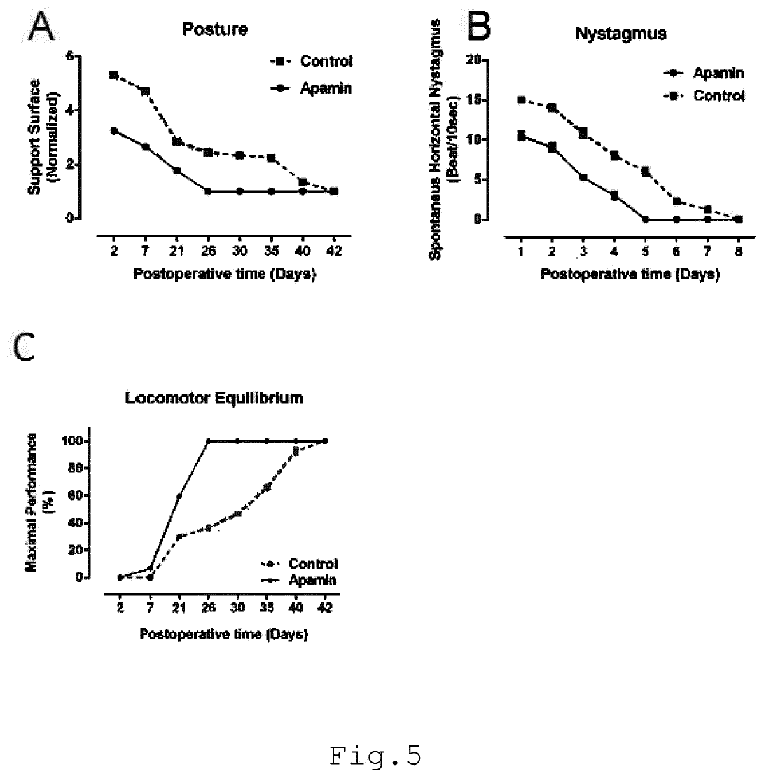

Posture function recovery: as shown in curves A, data recorded after vestibular deafferentation are related to individual references and normalized with respect to the preoperative values referred to unity (one being close to 50 cm²). Standard errors of the mean (S.E.M.) are reported as vertical lines. In four-footed animals standing erect, vestibular syndrome leads to an increased support surface delimited by the four paw pads. This parameter provides a good estimation of postural stability and recovery. It displays the tonic asymmetries of extensor and flexor muscles of the anterior and posterior paws that are induced by the vestibular deafferentation. Return to preoperative control values was faster for the UVN-apamin group (26 days) than the UVN-NaCl group (42 days) (p<0.0001) (FIG. 5).

Nystagmus: as shown in curves B, each data point represents the mean number of HSN quick phase movements in 10 s for 4 animals (five repeated measures per animal per sampling). Error bars represent S.E.M.

In particular, at the first post-UVN day, the frequency of the spontaneous nystagmus was 15 beats/10 sec in the UVN-NaCl and 11 beats/10 sec in the UVN-apamin groups, respectively. The number of eye beats declined significantly in these two experimental groups to reach control values at day 5 in the UVN-apamin group and at day 8 in the UVN-NaCl group (p<0.0001) (FIG. 5).

Locomotor balance recovery: as shown in curves C, the percent of the preoperative maximal performance (ordinates) is expressed as a function of the postoperative time in days (abscissae). S.E.M. are reported as vertical lines. In line with data of the posture function and the nystagmus, animals of the UVN-apamin group more quickly recovered their dynamic locomotor balance and crossed the rotating beam at their maximal performance (Max P.), at the 26th day after deafferentation. The cats of the UVN-NaCl group reached their Max P. 42 days after deafferentation (p<0.0001; FIG. 5).

Conclusion:

Antagonization of the apamine-sensitive SK channels significantly alters the time course of the vestibular syndrome induced by the UVN. Reductions of the oculomotor and posturolocomotor deficits are noticed from 24 h after the first apamin administration, while the effect persists well beyond the period of the drug application. Indeed, a significant reduction of the horizontal spontaneous nystagmus is noticed from the first administration of apamin and this effect significantly reduces the period of expression of the spontaneous nystagmus. It one must distinguish an immediate apamin effect on the static (posture surface and horizontal nystagmus parameters) and dynamic (locomotor balance function) vestibular deficits, from a persistent effect of apamin.

### Example 4: Effect of Apamine, AG525E1 and NS8593 Treatments on the Rat Unilateral Vestibular Neurectomy Model

Materials & Methods:

A unilateral vestibular neurectomy (UVN) was performed on adult female rats in order to induce unilateral vestibulopathy. Then, the small conductance calcium activated K⁺ channels antagonists apamin, AG525E1 and NS8593 were administered intraperitoneally to UVN animals in double blind conditions once a day for 4 days, i.e. during the acute phase of the syndrome. It was then assessed the severity of posturo-locomotor deficits using appropriate behavior tests for 10 days after UVN. Subjective scoring of the vestibular syndrome was carried out according to the method previously published (Péricat et al. 2017; Tighilet et al. 2017). According to this method, the severity of the vestibular syndrome of the injured animals was evaluated by subjectively assigning a score to various vestibular signs (VS) observed: —spontaneous or evoked rotation of the animal on its lateral axis (tumbling): score from 0 to 3 (0: absence of VS; 1: slight vestibular sign; 2: marked VS; 3 maximum VS)—spontaneous rotation or evoked from the animal according to a vertical axis (circling): score from 0 to 2 (0: absence of VS; 1: marked VS; 2 maximum VS)—repeated vertical movements of the head (bobbing): score from 0 to 1 (0: absence of VS; 1: presence of VS)—inclination of the head on the side of the lesion: score from 0 to 3 (see below)—inability to stand up to the wall: score from 0 to 1 (see below)—difficulty moving forward and/or alteration of the locomotor pattern: score from 0 to 4 (0: absence of VS; 1: slight vestibular sign; 2: evident VS; 3:marked VS; 4 maximum VS)—rotation of the body of the animal when undergoing vertical traction fast: score from 0 to 3 (see below). A total score was obtained for each animal by adding these different scores.

Apamin:

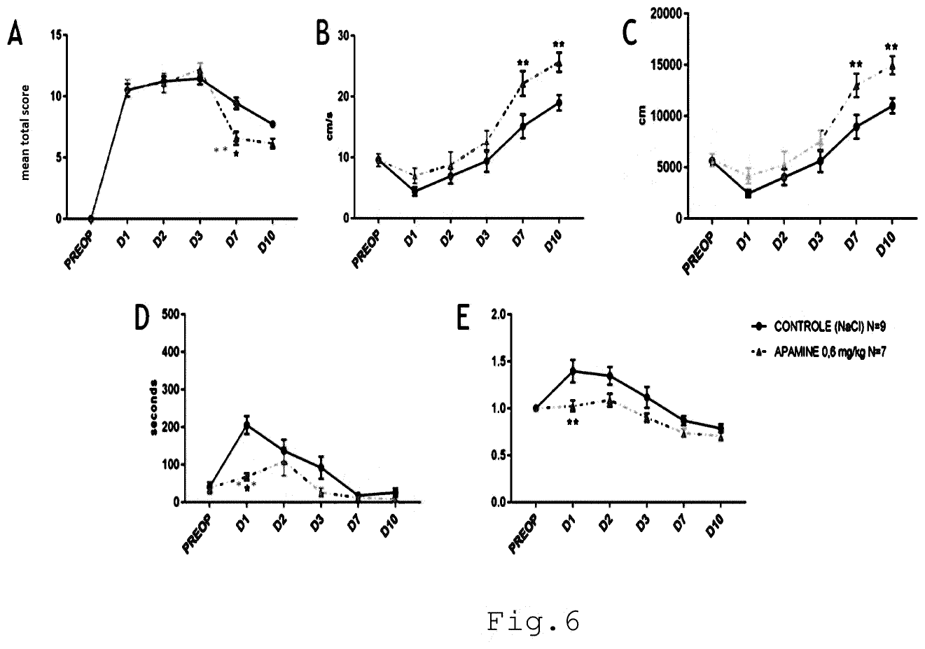

FIG. 6 shows the effects of an apamin treatment on vestibular syndrome severity over time (FIG. 6A), animal velocity (FIG. 6B), total distance covered (FIG. 6C), immobility time (FIG. 6D) and animal path shape (normalized meander) (FIG. 6E). In FIG. 6 “*” is indicative of a significant difference (p<0.05), “**” is indicative of very significant difference (p<0.01), and “***” in indicative of highly significant difference (p<0.001) compared to a negative control group, two-way ANOVA. As it appears on FIG. 6, 0.6 mg/kg of apamin that is administered after unilateral vestibular loss leads to a reduction of posturo-locomotor deficits resulting in faster attenuation of vestibular syndrome (statistically significant at day 7) (FIG. 6A), significant increase of animal velocity and distance covered at day 7 and day 10 (FIGS. 6B, 6C), reduced time of immobility and improvement of locomotion pattern at day 1 after neurectomy (FIGS. 6D, 6E).

AG525E1 and NS8593:

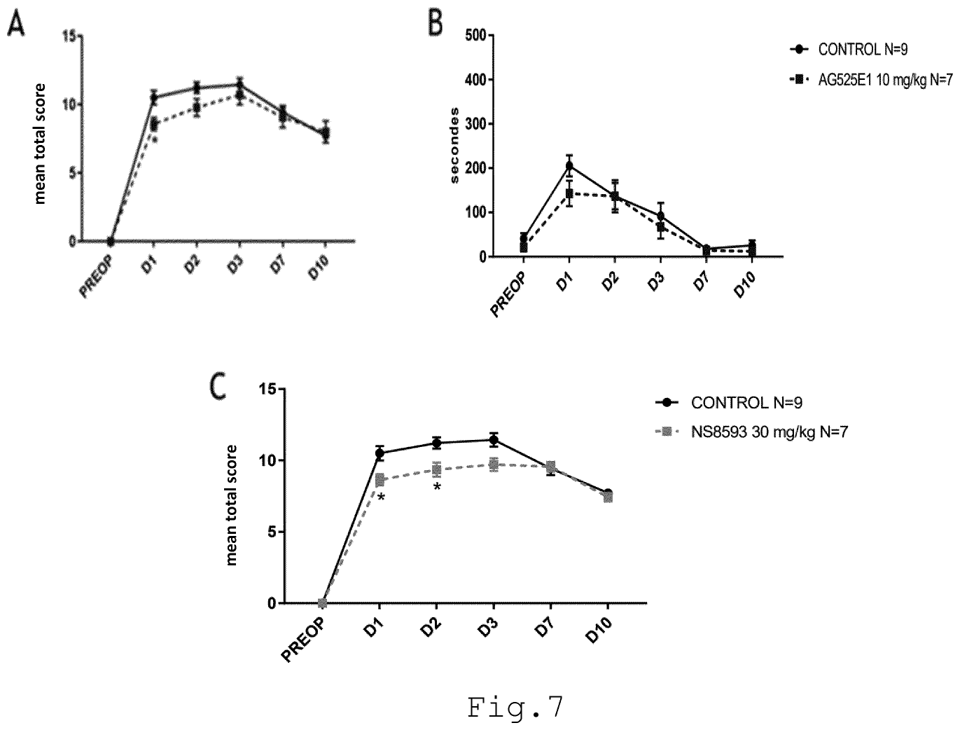

FIG. 7 shows the effects of AG525E1 (10 mg/kg) and NS8593 (30 mg/kg) treatments on vestibular syndrome severity (FIGS. 7A, 7C) and immobility time (FIG. 7B). In FIG. 7 “*” is indicative of a significant difference (p<0.05) compared to a negative control group, two-way ANOVA. It appears that administration of AG525E1 or NS8593, two other SK channel antagonists, induces a significant decreased vestibular syndrome at day 1 for AG525E1 (FIG. 2A) and at day 1 and day 2 for NS8593 (FIG. 2C). Administration of AG525E1 also leads to a tendency to reduce immobility time (FIG. 2B) caused by the UVN from day 1.

Discussion/Conclusion

Small conductance calcium activated K⁺ channels inhibitors administration during the acute phase of the vestibular syndrome, in particular apamin, but also other inhibitors such as AG525E1 and NS8593 provide significant antivertigo effect illustrated by significant reduction of the syndrome severity (Apamin, AG525E1, NS8593) and immobility time (apamin), increase of animal velocity (Apamin), total distance covered (apamin) and alteration of animal path shape-normalized meander (apamin).

The immediate modulatory effect of these inhibitors, in particular apamin, may result from a strong stimulatory action on the VSNs on the injured side, whose excitability is greatly reduced after UVN, with a less efficient action on the VSNs of the opposite side, whose excitability is already strongly stimulated by the removal of the inhibitory control of the ipsilateral VN. The antivertigo action seems thus to be the result of a reduction in the imbalance of activity between opposite VNs. Finally, conversely to a vestibulo-depressant action, aiming at reducing the imbalance between the opposing VNs by simultaneous inhibitory actions, the excitatory action of apamin reaches similar result, though exacerbating neuronal hyperexcitability. Acceleration of the vestibular compensation under apamin administration, observed already in the acute phase of the compensation and extending to its late phase is an illustration of such a phenomenon.

### REFERENCES

Xerri C, Lacour M. Compensation deficits in posture and kinetics following unilateral vestibular neurectomy in cats. The role of sensorimotor activity. Acta Otolaryngology (1980) 90(5-6): 414-24.

Lacour, M., Roll, J P., 1976. Modifications and development of spinal reflexes in the alert baboon (Papio papio) following an unilateral vestibular neurotomy. Brain res, 113(2), 255-269.

Mourre C, Hugues M, Lazdunski M. (1986). Quantitative autoradiographic mapping in rat brain of the receptor of apamin, a polypeptide toxin specific for one class of Ca2+-dependent K+ channels. Brain Res. 382:239-49.

Berman A. L. (1968). The brain stem of the cat. A cytoarchitechtonic atlas with stereotaxic coordinates. Madison Wis.: University of Wisconsin Press.

Tighilet, B., Trottier, S., Mourre, C., Lacour, M., 2006. Changes in the histaminergic system during vestibular compensation in the cat. J Physiol London, 573(3), 723-739.

Tighilet, B., Leonard, J., Lacour, M., 1995. Betahistine dihydrochloride treatment facilitates vestibular compensation in the cat. J Vestib Res, 5, 53-66.

Patkó T, Vassias I, Vidal P P, De Waele C. (2003) Modulation of the voltage-gated sodium- and calcium-dependent potassium channels in rat vestibular and facial nuclei after unilateral labyrinthectomy and facial nerve transection: an in situ hybridization study. Neuroscience. 117:265-80.

Brahim Tighilet, David Pericat, Alais Frelat, Yves Cazals, Guillaume Rastoldo, Florent Boyer, Olivier Dumas, Christian Chabbert, 2017, Adjustment of the dynamic weight distribution as a sensitive parameter for diagnosis of postural alteration in a rodent model of vestibular deficit. PLOS ONE.

1. A pharmaceutical composition comprising at least one inhibitor of small conductance calcium activated K+ channels of vestibular nuclei cells, and at least one pharmaceutically acceptable excipient, wherein the composition is effective in the treatment of a lesional vestibular disorder in a patient in need thereof.

2. The pharmaceutical composition according to claim 1, wherein the disorder is a lesional peripheral vestibular disorder.

3. The pharmaceutical composition according to claim 1, wherein the disorder is selected from the group consisting of vertigo, nystagmus, balance unsteadiness and loss of muscular tonus.

4. The pharmaceutical composition according to claim 1, wherein the inhibitor is apamin, ULC1684, tapamin, NS8593, tapamin-2, leiurotoxin I, POi, PO2 and PO5, tityus K, BmSKTx1, tubocurarine, atracurium, dequalinium, or AG525E1.

5. The pharmaceutical composition according to claim 4, wherein the inhibitor is apamin, NS8593 or AG525E1.

6. The pharmaceutical composition according to claim 5, wherein the inhibitor is apamin.

7. The pharmaceutical composition according to claim 6, wherein the composition is in a form adapted to administer apamin to the patient in an amount in a range of from 0.1 mg/kg and 0.5 mg/kg.

8. The pharmaceutical composition according to claim 7, wherein the composition is in a form adapted to administer apamin to the patient at a level of approximately 0.3 mg/kg.

9. The pharmaceutical composition according to claim 1, wherein the small conductance calcium activated K+ channels comprises three subunits SKI, SK2 and SK3 and wherein the inhibitor is effective to inhibit said channels in acting on at least one of said subunits.

10. The pharmaceutical composition according to claim 9, wherein the inhibitor is effective in acting on the SK2 subunit of the small conductance calcium activated K+ channels.

11. The pharmaceutical composition according to claim 1, wherein the composition is effective when administered between 30 min and 5 h after an insult.

12. (canceled)

13. The pharmaceutical composition according to claim 1, wherein the composition is in a form adapted to be administered by intraperitoneal route.

14. A method of treating a lesional vestibular disorder in a patient in need thereof, comprising administering to the patient an inhibitor of small conductance calcium activated K+ channels of vestibular nuclei cells, wherein the inhibitor is effective in the treatment of the lesional vestibular disorder in the patient.

15. The method according to claim 14, wherein the disorder is a lesional peripheral vestibular disorder.

16. The method according to claim 14, wherein the disorder is selected from the group consisting of vertigo, nystagmus, balance unsteadiness and loss of muscular tonus.

17. The method according to claim 14, wherein the inhibitor is apamin, ULC1684, tapamin, NS8593, tapamin-2, leiurotoxin I, POi, PO2 and PO5, tityus K, BmSKTx1, tubocurarine, atracurium, dequalinium, or AG525E1.

18. The method according to claim 17, wherein the inhibitor is apamin, NS8593 or AG525E1.

19. The method according to claim 18, wherein the inhibitor is apamin.

20. The method according to claim 19, wherein apamin is administered to the patient in an amount ranging from 0.1 mg/kg and 0.5 mg/kg.

21. The method according to claim 14, comprising administering a pharmaceutical composition comprising the inhibitor and at least one pharmaceutically acceptable excipient.
